(19) United States
(12) Patent Application Publication
(10) Pub. No.: US 2022/0265449 A1
(43) Pub. Date: Aug. 25, 2022

(54) STENT AND CORRESPONDING INSERTION DEVICE

(71) Applicant: EUROMEDICAL S.R.L., SAN ZENO NAVIGLIO (BS) (IT)

(72) Inventors: Ilario BONERA, BRESCIA (IT); Giorgio RAMORINO, SAN ZENO NAVIGLIO (BS) (IT)

(21) Appl. No.: 17/630,290

(22) PCT Filed: Jul. 28, 2020
(86) PCT No.: PCT/IB2020/057096

§ 371 Date: Jan. 26, 2022

(30) Foreign Application Priority Data
Jul. 29, 2019 (IT) 10 2019 000013206

(51) Int. Cl. A61F 2/94; A61F 2/95; A61F 2/848; A61F 2/04

(52) U.S. Cl. CPC A61F 2/94; A61F 2/95; A61F 2/848; A61F 2/04; A61F 2002/825

(57) ABSTRACT

A stent comprising a hollow tubular body, for instance made of polymeric material, extending along and about a longitudinal axis between two opposite open axial ends and having a substantially cylindrical lateral wall delimiting a longitudinal inner conduit; the body comprising a plurality of deformable sections arranged in succession along the axis; each section comprises a plurality of longitudinal portions of the lateral wall and a plurality of radial cuts formed through the lateral wall and which separate the longitudinal portions from one another.

### CROSS-REFERENCE TO RELATED APPLICATIONS

This patent application claims priority from Italian patent application no. 102019000013206 filed on Jul. 29, 2019, the entire disclosure of which is incorporated herein by reference.

### TECHNICAL FIELD

The present invention relates to a stent and a device for inserting such stent.

### BACKGROUND ART

The term stent generally refers to an expandable prosthesis which is introduced in a body duct or lumen organ to restore its patency when this is reduced or impaired.

Stents, for example, are used in blood vessels (arteries and veins) and in other tubular structures such as the bile duct, oesophagus, trachea, ureter, etcetera.

The stent is inserted into the duct to be treated, normally by a catheter, in a minimum size retracted form; once in position, the stent is expanded and takes on a substantially tubular expanded shape of use.

Despite various types of stents and corresponding insertion devices are known, there still seems to be room for improvement in the prior art stents and the corresponding insertion devices.

In particular, various types of stents are made of metal materials, possibly shape memory materials, and are thus particularly complicated and expensive to make, even generally having relatively great sizes especially if wide dilations are required.

The use of different materials, in particular polymeric materials, has not been, for the time being, tangibly applied.

Furthermore, the prior art stents cannot normally be progressively expanded and it is not possible to decide the degree of the stent dilation during the implantation step.

Further, the known stents cannot normally be recovered, namely once having been dilated they can no longer be retracted and moved, for instance for correcting a possible imprecision in the implantation position.

### DISCLOSURE OF INVENTION

It is an object of the present invention to provide a stent and a corresponding operating device for insertion thereof that overcome the above noted drawbacks of the prior art.

The present invention thus relates to a stent and a device for inserting a stent as defined in the enclosed claims 1 and, respectively, 7.

Preferred auxiliary features are defined in the dependent claims.

The stent of the invention is easy to make and use as well as fully efficient and reliable.

The stent is preferably made of polymeric material (plastic), thus being of particularly easy and cheap construction.

Furthermore, the stent of the invention has a progressive dilation and can be progressively dilated remaining stable in each configuration taken on: it is thus possible to decide the degree of dilation of the stent while implanting it.

The stent can also be recovered, as it has a reversible dilation: once having been dilated, the stent can always be taken back to the initial retracted configuration, for instance to be repositioned.

In particular, the stent can be recovered in a sectional way, allowing to select the number of recover sections.

The stent can also be provided with one or more radiopaque markers, at a distal, proximal position and/or at an intermediate position between the distal and proximal position.

### BRIEF DESCRIPTION OF THE DRAWINGS

Further features and advantages of the present invention will become clear from the description of the following non-limiting embodiments, with reference to the figures of the accompanying drawings, wherein:

FIG. 1 is a perspective schematic, partially sectioned view of a stent and corresponding insertion device according to the invention;

FIG. 2 is a longitudinal partially sectioned view of the device of FIG. 1;

FIG. 3 is an exploded perspective schematic view of the device of FIG. 1;

FIG. 4 is an enlarged scale perspective view of the stent according to the invention;

FIG. 5 is an enlarged scale perspective view of a component of FIG. 1;

FIGS. 6 and 7 are enlarged scale respective views of the details highlighted in FIG. 2;

FIGS. 8-18 schematically show the functioning of the device of the invention, represented in a side view in several positions of use (FIGS. 8-9, 11, 13, 15, 17-18) and by certain enlarged scale and longitudinal sectioned details (FIGS. 10, 12, 14, 16).

### BEST MODE FOR CARRYING OUT THE INVENTION

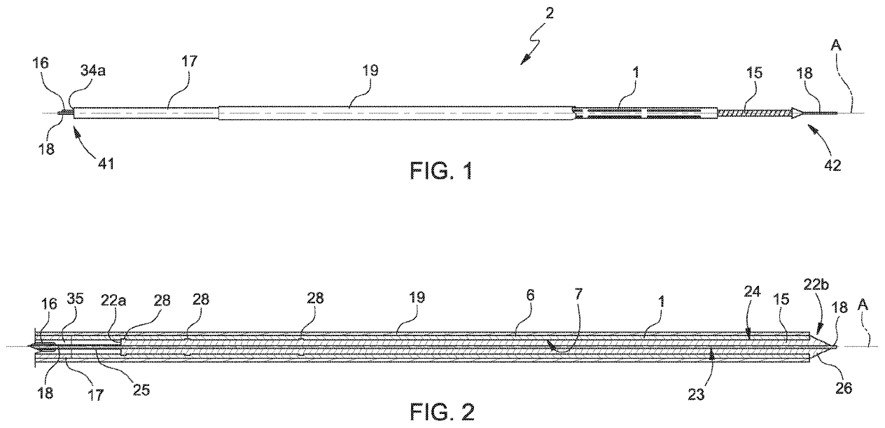

In FIGS. 1-3 a stent 1 is shown together with an insertion device 2 for inserting the stent 1 in a body duct or lumen organ.

The stent 1 is illustrated in a minimum size closed configuration, to be used while inserting and positioning the stent 1 by the device 2.

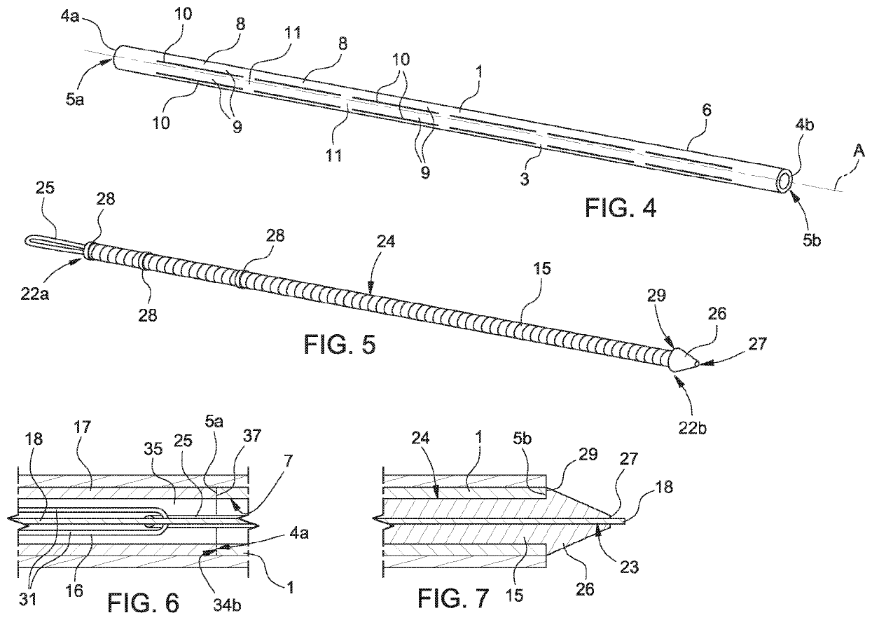

Referring specifically also to FIG. 4, the stent 1 has a hollow tubular body 3 extending along and about a longitudinal axis A and for example substantially cylindrical.

According to a preferred aspect of the invention, the body 3 of the stent 1 is made of polymeric material (plastic), such as a polymeric elastomer material (such as, though not necessarily, selected among silicones, polyurethanes, TPE and similar) or a thermo-plastic polymeric material belonging to the biomedical polymeric category (such as, though not necessarily polyamide, acetalic resin, polypropylene, polyethylene, etcetera).

The body 3 extends between two opposite open axial ends 4a, 4b, provided with respective annular end edges 5a, 5b, and has a substantially cylindrical lateral wall 6 delimiting a longitudinal inner conduit 7 extending between the ends 4a, 4b.

The body 3 comprises a plurality of deformable sections 8 arranged in succession along the axis A.

Each section 8 comprises a plurality of longitudinal portions 9 of the lateral wall 6 and a plurality of radial cuts 10 formed through the lateral wall 6 and separating the longitudinal portions 9 from one another.

The cuts 10 of each section 8 are in particular substantially parallel to one another and angularly staggered about the axis A.

The cuts 10 are for instance in the form of slots longitudinally elongated parallel to the axis A.

Each section 8 has two or more cuts 10 and is axially delimited by a pair of annular portions 11 of the lateral wall 6.

The annular portions 11 of the lateral wall 6 thus join to one another the sections 8 and separate the cuts 10 of the various sections 8 that axially succeed on the body 3.

In the illustrated non-limiting example, though not necessarily, the cuts 10 of the various sections 8 are aligned between each other and have a similar length. It must be understood that the cuts 10 can be differently arranged and organised and be placed at a different pitch and/or orientation from what herein disclosed and illustrated for merely exemplary purposes.

Referring again to FIGS. 1-3, the device 2 for inserting the stent 1 extends along the axis A and comprises:

an operating member 15, an actuation wire 16, a pusher 17, a guide wire 18 and optionally a cover sheath 19.

The operating member 15, illustrated in greater detail in FIG. 5, has an internally hollow flexible tubular body, made for instance (though not necessarily) of metal material. For instance, the operating member 15 has a corrugated or spiral structure tubular body.

The operating member 15 extends longitudinally along the axis A between opposite open longitudinal ends 22a, 22b and has an inner longitudinal conduit 23 passing entirely through the operating member 15 between the ends 22a, 22b.

The operating member 15 has an outer lateral surface 24, optionally (depending also on the constructive structure of the operating member 15) wavy or coiled.

The end 22a is provided with an eyelet 25 projecting from the end 22a along the axis A; the opposite end 22b is provided with a pilot head 26, radially projecting from the surface 24 and staggered towards the end 22b or substantially conical or truncated-cone. The head 26 ends with a front opening 27 in communication with the conduit 23.

The surface 24 has a series of annular projections 28, radially projecting from the surface 24 and longitudinally spaced apart from one another along the operating member 15. In particular, the operating member 15 has a first projection 28 at the end 22a and further successive projections 28 along the axis A.

Projections 28 extend from the surface 24 to contact an inner lateral surface of the stent 1, when the operating member 15 is housed in the conduit 7 of the stent 1, or of the pusher 17, when the operating member 15 slides outside the conduit 7 of the stent 1 inside the pusher 17, as will be clearly explained hereinafter.

In the device 2 ready for use, i.e. for introducing the stent 1 in a patient (FIG. 1), the operating member 15 in arranged inside the stent 1 and in particular inside the conduit 7 of the stent 1, i.e. the stent 1 is fitted about the operating member 15 and surrounds it radially outside.

The eyelet 25 and the head 26 project axially, at least partially, outside the conduit 7 from respective ends 4a, 4b of the stent 1.

In particular, the eyelet 25 projects axially, at least partially, from the end 4a beyond the edge 5a (FIG. 6); and the head 26 projects from the end 4b beyond the edge 5b and axially abuts against the edge 5b with an annular shoulder 29 facing the edge 5b of the stent 1 (FIG. 7).

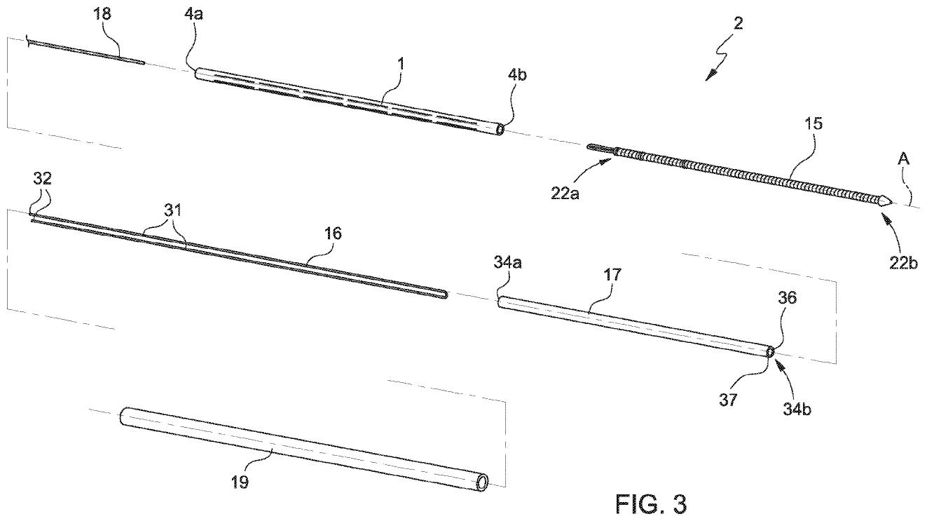

Referring specifically to FIGS. 3 and 6, the actuation wire 16 is a wire, such as a metal wire, that is U-folded so as engage the eyelet 25 of the operating member 15; in particular, the actuation wire 16 is inserted into the eyelet 26 and U-folded so as to have a pair of parallel arms 31 exiting from the eyelet 25 and ending with respective ends 32.

Referring specifically to FIGS. 3 and 6, the pusher 17 is a tubular element, for instance of metal or polymeric material (plastic), extending between two opposite open axial ends 34a, 34b.

The pusher 17 is provided with an inner longitudinal conduit 35 extending from the ends 34a, 34b.

The pusher 17 is axially aligned to the stent 1 along the axis A and has a front edge 36, placed at the end 34b, defining an axial shoulder 37 axially abutting against the edge 5a of the stent 1.

The guide wire 18 is for guiding the insertion of the device 2 into the patient's organ where the stent 1 must be implanted; for instance, the guide wire 18 is a metal wire capable of transmitting axial forces (along a wire longitudinal axis) in opposite directions.

The guide wire 18 passes through the entire device 2 along the axis A: in particular, the guide wire 18 is inserted in the conduit 23 of the operating member 15 (axially projecting from the opposite ends 22a, 22b of the operating member 15), in the conduit 7 of the stent 1, and in the conduit 35 of the pusher 17; the guide wire 18 projects outside from the end 34a of the pusher 17 with the arms 31 of the actuation wire 16; and from the end 22b of the operating member 15 through the opening 27.

The (optional) cover sheath 19, for example made of polymeric material, is placed about the stent 1 and at least a portion of the pusher 17.

Once the device 2 is assembled (FIG. 1) the device 2 extends along the axis A between a proximal end 41, defined by the end 34a of the pusher 17 and from which a first end of the guide wire 18 and two arms 31 placed side by side of the actuation wire 16 project outside; and a distal end 42, defined by the end 22b of the actuating member 15 and from which a second end of the guide wire 18 projects outside.

The stent 1 is implanted by of the device 2, which can be operated by any instrument of the known type for this kind of operations, which does not pertain to the present invention and not herein described nor illustrated for ease of simplicity.

Once the stent 1 is mounted on the device 2, in the assembled configuration shown in FIG. 1, the device 2 is introduced in the hollow organ where the stent 1 must be implanted.

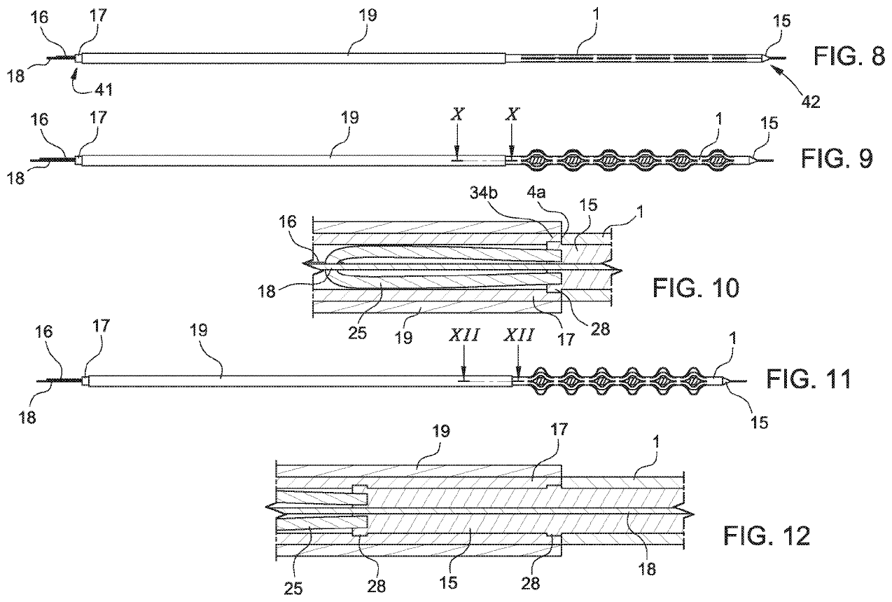

Once in position, the stent 1 is extracted from the cover sheath 19 (if this is present) acting on the pusher 17 at the proximal end 41 of the device 2 (FIG. 8). In particular, the pusher 17 is moved in an axial direction along the axis A towards the distal end 42 of the device 2; the shoulder 37 of the pusher 17 pushes the edge 5a of the stent 1 and thus pushes the stent 1 out of the cover sheath 19.

The stent 1 is thus radially dilated (FIG. 9) acting on the actuation wire 16, which is pulled backward, i.e. moved away from the proximal end 41 of the device 2, while the pusher 17 is maintained axially fixed: the actuation wire 16, being hooked to the eyelet 25, moves the operating member 15 backward acting on the end 4b of the stent 1, by the head 26 cooperating with the edge 5b of the stent 1.

The end 4b of the stent 1 moves back, upon being pushed by the shoulder 29 of the head 26 of the operating member 15; as the opposite end 4a is axially blocked by the edge 36 (by the shoulder 37) of the pusher 17 contacting the edge 5a, the stent 1 shortens along the axis A and deforms radially, thanks to the cuts 9 that allow the deformation of the sections 8 and in particular of the portions 9.

The axial squeezing of the stent 1 (i.e. the movement of the opposite ends 4a, 4b thereof close to one another)thereby causes the radial expansion of the sections 8 which take on an arched shape. The stent 1 takes on a wavy conformation with a number of waves defined by the sections 8.

The sections 8 are plastically deformable, i.e. they stay in the deformed shape taken on upon being stressed (in particular, after axial compression of the body 3 of the stent 1) even after been stressed; the overall sections 8 and stent 1 return to the initial position (i.e. in the minimum size closed configuration of the stent 1) if submitted to a contrary stress, namely if the body 3 of the stent 1 is submitted to elongation, moving the ends 4a, 4b away from each other).

The operating member 15 is axially moved relative to the pusher 17, which remains fixed; the axial position along the axis A of the operating member 15 relative to the pusher 17 can be adjusted by projections 28 which the operating member 15 is provided with.

In fact (FIG. 10), when a projection 28 exits from the end 4a of the stent 1 and contacts the pusher 17, for instance engaging an annular seat formed on an inner lateral surface of the pusher 17 at the end 34b of the pusher 17, the user operating the device 2 perceives the position snap of the operating member 15 and knows where the operating member 15 is in relation to the stent 1 as well as the dilation degree of the stent 1.

Continuing the movement of the operating member 15 to the subsequent projection 28 the stent 1 continues to deform dilating progressively (FIGS. 11-12).

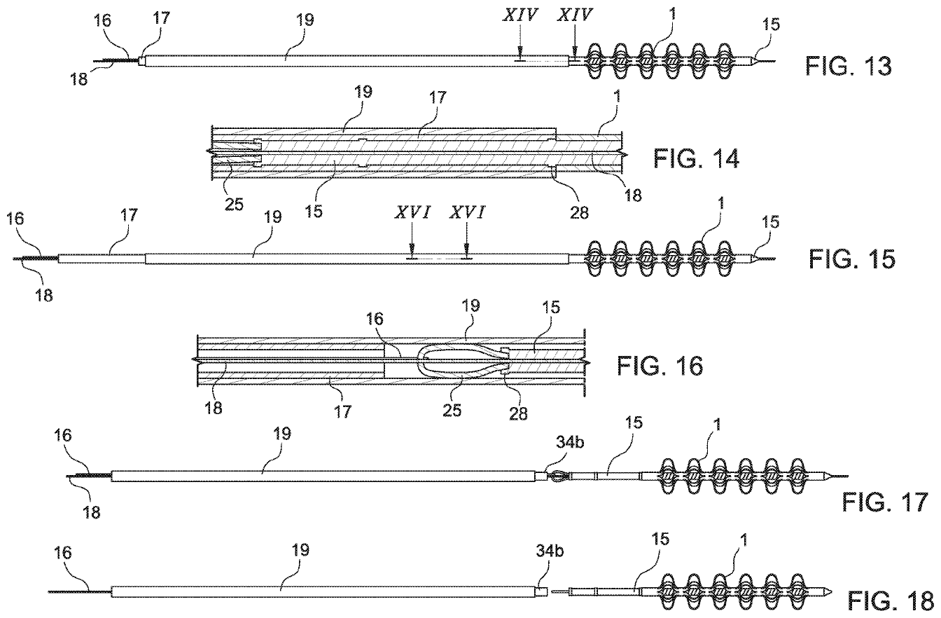

When the last projection 28 exits from the stent 1, the stent 1 is completely expanded (FIGS. 13-14).

Now, the pusher 17 is moved backward along the axis A (i.e. it is moved away from the distal end 42 of the device 2) and is moved out of the operating member 15 (FIGS. 15-16). The eyelet 25 of the operating member 15 exits from the pusher 17 and it is free to deform taking on the size allowed by the cover sheath 19.

The pusher 17 is then again moved forward along the axis A, i.e. towards the distal end 42 of the device 2, until it projects outside with its end 34b from the cover sheath 19 and thus also pushes the operating member 15 out of the cover sheath 19 (FIG. 17).

Removal is carried out of the guide wire 18, which is simply pulled backwards and moved out of the conduit 7 of the stent 1, from the conduit 23 of the operating member 15, and from the conduit 35 of the pusher 17; and of the actuation wire 16, which is extracted pulling one of the ends 32 and keeping the other end 32 free, in order to clear the eyelet 25 (FIG. 18).

By contrast the operating member 15 remains inside the stent 1.

Finally, it must be understood that changes and variants can be brought to the herein described stent and insertion device without departing from the scope of the enclosed claims.

1. A stent (1) comprising a hollow tubular body (3) extending along and about a longitudinal axis (A) between two opposite open axial ends (4a, 4b) and having a substantially cylindrical lateral wall (6) delimiting a longitudinal inner conduit (7); the body (3) comprising a plurality of deformable sections (8) arranged in succession along the axis (A); each section (8) comprising a plurality of longitudinal portion (9) of the lateral wall (6) and a plurality of radial cuts (10) formed through the lateral wall (6) and which separate the longitudinal portions (9) from one another; characterized in that the sections (8) are plastically deformable as a consequence of axial compression and expansion of the body (3) remaining in a deformed shape taken on upon being stressed after axial compression of the body (3) even after been stressed, and returning to a minimum size closed configuration if the body (3) is submitted to elongation, so that the stent (1) has a progressive dilation and can be progressively dilated remaining stable in each configuration taken on and the stent (1) has a reversible dilation and once having been dilated can be taken back to the initial retracted configuration.

2. A stent according to claim 1, wherein the cuts (10) of each section (8) are substantially parallel to one another and angularly staggered about the axis (A) with respect to one another.

3. A stent according to claim 1, wherein the cuts (10) are in the form of slots longitudinally elongated parallel to the axis (A).

4. A stent according to claim 1, wherein each section (8) has two or more cuts (10) and is axially delimited by a pair of annular portions (11) of the lateral wall (6) which join to one another the sections (8) and separate the cuts (10) of the respective sections (8).

5. (canceled)

6. A stent according to claim 1, wherein the stent (1) is made of polymer material.

7. An insertion device (2) for inserting a stent (1) according to claim 1 in a body duct or lumen organ; the device (2) extending along the axis (A) between a proximal end (41) and a distal end (42) and comprising: said stent (1); and an operating member (15) and a pusher (17), having respective opposite axial shoulders (29, 37) axially cooperating in contact with respective opposite axial ends (4a, 4b) of the stent (1) and movable with respect to one another along the axis (A) to axially compress the stent (1) and cause deformation of the deformable sections (8) of the stent (1).

8. A device according to claim 7, wherein the pusher (17) is defined by a tubular element axially aligned to the stent (1) along the axis (A) outside the stent (1) and cooperates with a first end (4a) of the stent (1) via a first shoulder (37) defined by an annular front edge (36) of the pusher (17); and wherein the operating member (15) is housed inside the conduit (7) of the stent (1) and projects outside a second end (4b) of the stent with a head (26) provided with a second shoulder (29) facing and cooperating with said second end (4b) of the stent (1).

9. A device according to claim 7, comprising an actuation wire (16) engaging an eyelet (25) of the operating member (15) to axially move the operating member (15) towards the proximal end (41) of the device (2).

10. A device according to claim 9, wherein the actuation wire (16) is inserted in the eyelet (26) and U-folded so as to have a pair of parallel arms (31) exiting from the eyelet (25) and ending with respective ends (32).

11. A device according to claim 7, wherein the operating member (15) has a flexible internally hollow tubular body.

12. A device according to claim 7, wherein the operating member (15) is provided with a series of annular projections (28), radially projecting from an outer lateral surface (24) of the operating member (15) and longitudinally spaced apart from one another along the operating member (15) to contact radially an inner lateral surface of the stent (1), when the operating member (15) is housed in the conduit (7) of the stent (1), or of the pusher (17), when the operating member (15) slides outside the conduit (7) of the stent (1) and inside the pusher (17).

13. A device according to claim 7, wherein the operating member (15) has a corrugated or spiral structure tubular body, preferably made of metal material.

14. A device according to claim 7, comprising a guide wire (18) arranged along the axis (A) through the device (2) and exiting from said proximal end (41) and from said distal end (42) of the device (2); the guide wire (18) being housed in respective conduits (7, 23, 35) formed through the stent (1), the operating member (15) and the pusher (17).
